(19) United States
(12) Patent Application Publication
(10) Pub. No.: US 2022/0030979 A1
(43) Pub. Date: Feb. 3, 2022

(54) DISPOSABLE SEMI-HARDENED FACE MASK ATTACHED WITH GLASSES

(71) Applicant: Sherman Nettles, Clarksville, TN (US)

(72) Inventor: Sherman Nettles, Clarksville, TN (US)

(21) Appl. No.: 17/444,036

(22) Filed: Jul. 29, 2021

Related U.S. Application Data
(60) Provisional application No. 63/058,063, filed on Jul. 29, 2020.

(51) Int. Cl. A41D 13/11

(52) U.S. Cl. CPC A41D 13/1184; A41D 13/1161

(57) ABSTRACT

A disposable semi-hardened face mask attached with glasses is disclosed. The disposable face mask is formed of a material and shaped to be disposed across a portion of the face of a wearer. The glasses comprise a frame with bendable temple arms and lenses, wherein the frame is curved to ensure maximum fit and configured to be comfortably worn by the wearer for protecting eyes. The glasses further comprise an interior seal for preventing entry of airborne pathogens that may pass through the eyes from entering into the body. The glasses are detachably secured at the bottom to the disposable face mask using fastening members, which are placed on an outer surface at a top portion of the mask. The disposable face mask attached with the glasses could maintain a sealed fit around the mouth, nose, and eyes to prevent the entry of airborne pathogens.

### CROSS-REFERENCE TO RELATED APPLICATIONS

This patent application claims priority to provisional patent application No. 63/058,063 filed Jul. 29, 2020, and titled “Total Virus Protection.” The subject matter of said is hereby incorporated by reference in its entirety.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention generally relates to a disposable face mask adapted for engagement with an eyewear device. More particularly, the present invention relates to an improved sealing face mask with a sealed glasses system used to protect a wearer against any exposure to airborne pathogens and maintain a sealed fit over the mouth, nose, and eyes.

### BACKGROUND

A face mask is a protective and/or hygienic face protector. The face mask is personal protective equipment worn by any individual. It prevents airborne transmission of infections between people by blocking the movement of pathogens such as primarily bacteria and viruses, shed in respiratory droplets and aerosols into and from a wearer's mouth and nose. Therefore, it is essential to provide a mask that can protect people to maintain health from environmental pollution.

Previously, the mask was majorly used to execute the function in the prevention of inhaling pollution in places such as industrial sites. Also, the mask was used to block the cold during the winter season. Nowadays, the mask is worn to prevent the wearer from being exposed to pollution or germs from others or respiratory diseases due to gas, or ultraviolet rays according to use.

The mask covers the face to play the key role of fulfilling safety from pollutants such as cold, ultraviolet rays, or the pollution environment. In addition, many individuals in modern society are putting effort into executing outdoor activities and outdoor exercises to find the vitality of life or maintain health. Further, people in cities and other urbanized areas experience considerable human congestion at maximum capacity. As a result, individuals on the street, subway train, cars, and other local areas may be forced to be in physical contact with each other in their daily life. Therefore, there may be a chance of transmission of pathogens such as infectious disease, bacteria, allergens, and/or other undesirable human-produced biologic material in such high-density areas.

Many individuals choose to proactively wear protective hygienic face masks to guard against inhaling undesirable biologic material expelled by nearby individuals through sneezing or coughing, and/or environmental-related contaminants, for example, smoke, other forms of industrial air pollution, and particulate matter. Such masks cover only the wearer's mouth and nose, reducing only a minimal chance of exposure to airborne pathogens. However, the eyes are often left uncovered unprotected.

The eyeglasses or eyewear devices with an interior seal will offer the protection to help reduce the number of pathogens that may pass through the eyes from entering the body. However, such readily available and mass-produced face masks may not be suitably equipped to attach, adhere, and/or otherwise combine the body of the face mask with eyewear, such as sunglasses. Thus, wearing sunglasses in combination with traditional face masks may require a wearer or user to choose whether to place the lenses of the glasses above the face mask or to pull the face mask above the lenses, to form an interface between the lenses of the glasses and the face mask. Further, for the users that wear glasses, the hot breath inside the mask is not able to be discharged to the front due to the mask. The air is discharged to the nose and the part where the nose and cheek meet to make the glasses foggy. Therefore, the sight of the user becomes blurry.

Due to the above-mentioned drawbacks, there is a need for a comfortable, relatively inexpensive, readily available disposable semi-hardened face mask adapted to combine and/or attach easily via a foam layer with eyeglasses.

### SUMMARY

The present invention discloses a face mask adapted for engagement with an eyewear device. Further, the present invention discloses an improved sealing face mask with a sealed glasses system configured to protect a wearer against any exposure to airborne pathogens and maintain a sealed fit over the mouth, nose, and eyes.

In one embodiment, a disposable semi-hardened face mask attached with glasses is disclosed. In one embodiment, the disposable semi-hardened face mask provides a sealed glasses system with an improved sealing face mask configured to avoid any exposure of the wearer's mouth, nose, and/or eyes to the airborne pathogens. In one embodiment, the disposable semi-hardened face mask attached with glasses is used to protect the wearer against any exposure of mouth, nose, and eyes to airborne pathogens, dust, germs, bacteria, virus, and other harmful infectious materials.

In one embodiment, the disposable semi-hardened face mask attached with glasses comprises a disposable face mask and a pair of glasses or glasses. In one embodiment, the pair of glasses is a sealed glasses system. In one embodiment, the face mask is a disposable face protector. In one embodiment, the face mask is detachably secured to the glasses configured to maintain a sealed fit over the mouth, nose, and eyes of the wearer.

In one embodiment, the disposable face mask is formed of a material and shaped to be disposed across a portion of the face of the user in alignment with and surrounding the nose and mouth of the user. In one embodiment, the disposable face mask is made of a standard 3-ply cotton material. In one embodiment, the disposable face mask has one or more vents for allowing exhaled air to easily escape from both sides of the disposable face mask, thereby preventing fogging of glasses due to moist and exhaled air that escapes from behind the disposable face mask. In one embodiment, the vents are at least any one of lightweight plastic or metal vents.

In one embodiment, the pair of glasses has a frame with bendable temple arms and lenses. In one embodiment, the frame is curved to ensure maximum fit and configured to be comfortably worn by the wearer to protect their eyes. In one embodiment, the glasses comprise an interior seal for preventing entry of the airborne pathogens that may pass through the eyes from entering into the body. In one embodiment, the glasses are trimmed around the lens region with at least any one of foam or a rubber material that will conform to the user's face.

In one embodiment, the disposable semi-hardened face mask attached with glasses further comprises one or more fastening members, which are placed on an outer surface at a top portion of the disposable face mask. In one embodiment, the glasses are secured at the bottom to the disposable face mask using a hook and loop fastener (Velcro®). In one embodiment, the fastening member is configured to detachably secure the glasses at the bottom to the disposable face mask, thereby forming and maintaining a sealed fit around the mouth, nose, and eyes to protect the wearer.

In one embodiment, the sealing of glasses to the disposable face mask prevents entry of airborne germs, bacteria, and viral and bacterial pathogens which can be transmitted from human to human or animal to human or human to animal through saliva, nasal fluid or inhalation. In one embodiment, the fastening member is at least any one of double-sided adhesive sticky tape and a hook and loop fastener (Velcro®).

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The foregoing summary, as well as the following detailed description of the invention, is better understood when read in conjunction with the appended drawings. For illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows a perspective view of a disposable semi-hardened face mask attached with glasses worn by a user, according to one embodiment of the present invention.

FIG. 2 shows a perspective view of the disposable semi-hardened face mask detachably secured to the glasses, according to one embodiment of the present invention.

FIG. 3 shows a top view of the disposable face mask detachably secured to the glasses in one embodiment of the present invention.

FIG. 4 shows an enlarged view of the disposable face mask provided with fastening members, for example, double-adhesive sticky tapes in one embodiment of the present invention.

### DETAILED DESCRIPTION

The present invention is best understood by reference to the detailed figures and description set forth herein.

It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

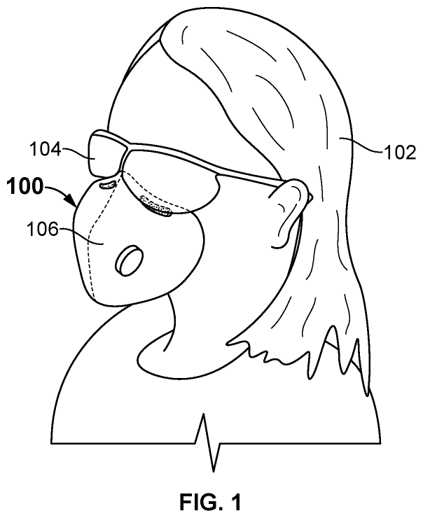

Referring to FIG. 1, a disposable semi-hardened face mask attached with glasses 100 worn by a user or wearer 102 in one embodiment of the present invention is disclosed. In one embodiment, the disposable semi-hardened face mask attached with glasses 100 provides a sealed glasses system with an improved sealing face mask. In one embodiment, the disposable semi-hardened face mask attached with glasses 100 comprises a pair of glasses 104 and a face mask 106. In one embodiment, the pair of glasses 104 is a sealed glasses system. In one embodiment, the face mask 106 is a disposable face protector. In one embodiment, the face mask 106 is detachably secured to the glasses 104 configured to maintain a sealed fit over the mouth, nose, and eyes. In one embodiment, the disposable semi-hardened face mask attached with glasses 100 is used to protect the wearer 102 against any exposure of mouth, nose, and eyes to airborne pathogens, dust, germs, bacteria, virus, and other harmful infectious materials.

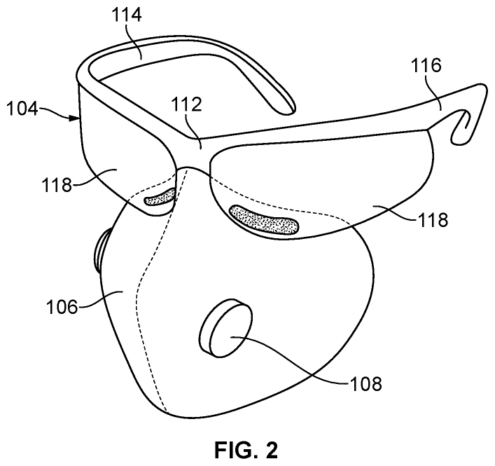

Referring to FIG. 2, a perspective view of the disposable semi-hardened face mask attached with glasses 100, according to one embodiment of the present invention. In one embodiment, the disposable semi-hardened face mask attached with glasses 100 comprises a disposable face mask 106 and a pair of glasses or glasses 104.

In one embodiment, the disposable face mask 106 is formed of a material and shaped to be disposed across a portion of the face of the user 102 in alignment with and surrounding the nose and mouth of the user 102. In one embodiment, the disposable face mask 106 is made of a standard 3-ply cotton material. In one embodiment, the disposable face mask 106 has one or more vents 108 for allowing exhaled air to easily escape from both sides of the disposable face mask, thereby preventing fogging of glasses 104 due to moist and exhaled air that escapes from behind the disposable face mask 106. In one embodiment, the vents 108 are at least any one of lightweight plastic or metal vents.

In one embodiment, the pair of glasses 104 has a frame 112 with bendable temple arms (114 and 116) and lenses 118. In one embodiment, the frame 112 is curved to ensure maximum fit and configured to be comfortably worn by the wearer 102 to protect eyes. In one embodiment, the glasses 104 comprise an interior seal for preventing entry of the airborne pathogens that may pass through the eyes from entering into the body. In one embodiment, the glasses 104 are trimmed around the lens region with at least any one of foam or a rubber material that will conform to the user's face.

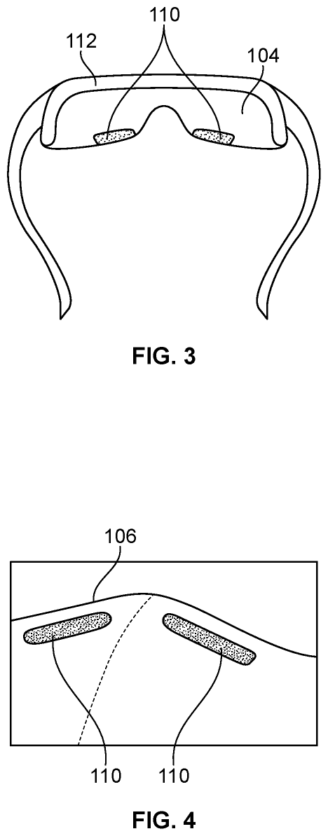

In one embodiment, the disposable semi-hardened face mask attached with glasses 100 further comprises one or more fastening members 110 (shown in FIG. 3) placed on an outer surface at a top portion of the disposable face mask 106. In one embodiment, a bottom portion of the glasses are configured to receive at least one flange extending radially outward from a top portion of the disposable face mask such that the disposable face mask 106 forms a first seal around the mouth and nose, and the glasses 104 form a second seal around the eyes. In one embodiment, the glasses 104 are secured at the bottom to the disposable face mask 106 using a hook and loop fastener (Velcro®). In one embodiment, the fastening members 110 are configured to detachably secure the glasses 104 at the bottom to the disposable face mask 106, thereby forming and maintaining a sealed fit around the mouth, nose, and eyes to protect the wearer 102. In one embodiment, the sealing of glasses 104 to the disposable face mask 106 prevents entry of airborne germs, bacteria, and viral and bacterial pathogens, which can be transmitted from human to human or animal to human or human to animal through saliva, nasal fluid or inhalation. In one embodiment, the fastening members 110 are at least any one of, but are not limited to, double-sided adhesive sticky tape and a hook and loop fastener (Velcro®).

Referring to FIG. 3, a top view of the disposable face mask 106 detachably secured to the glasses 104 in one embodiment is disclosed. In one embodiment, the disposable face mask 106 is detachably secured to the glasses 104 using the fastening members 110. The disposable face mask attached to the glasses 100 is configured to allow the users to easily attach and detach the disposable face mask 106 to the glasses 104 for full protection and enable the users to simply replace the disposable face mask 106 when needed via the adhesive or double-sided adhesive sticky tape and a hook and loop fastener (Velcro®). In one embodiment, the disposable face mask 106 and glasses 104 in combination form a seal around the mouth, nose, and eyes to protect the wearer 102 (as shown in FIG. 1).

In one embodiment, the disposable face mask 106 includes a mounting member that is configured to interchangeably mount either a single strap or two straps to the disposable face mask to establish a good seal around the user's nose and mouth. In one embodiment, at least one strap includes a buckle that is configured for adjusting the length of the strap and for fitting various head sizes to achieve maximum comfort and adaptability.

The glasses 104 further include foam to maintain comfort and receive a durable acid tape adhesive to connect the disposable face mask 106. The disposable face mask 106 is designed to be changed as often as needed, and the glasses 104 are designed to be kept. In one embodiment, the seal also around the glasses frame 112 may be changed as often as needed, which will keep both the glasses 104 and filters free of contamination of any pathogen. Users could easily attach the disposable face mask 106 to the glasses 104 and form the seal around the nose and mouth for inhalation protection. Users can wear both disposable face mask 106 and glasses 104 to keep themselves safe while in public places. The mask 106 simply detaches from the glasses 104 for allowing the user to change the contaminated mask with a new mask.

In one embodiment, the bottom of glasses 104 should be trimmed in a clear or black, thin hook and loop tape (Velcro®), or double-adhesive sticky tapes for detachably securing the disposable face mask 106 to the glasses 104. In one embodiment, the tops of the masks could be trimmed with a clear or black hook and loop tape (Velcro®), or double-adhesive sticky tapes for detachably securing the disposable face mask 106 to the glasses 104.

The disposable face mask attached to the glasses 100 is configured to protect the wearer against any exposure to airborne pathogens. The disposable face mask attached to the glasses 100 could form and maintain a sealed fit around the user's mouth, nose, and eyes to protect the wearer and prevent the entry of airborne germs, bacteria, and viral and bacterial pathogens which can be transmitted from human to human or animal to human or human to animal through saliva, nasal fluid or inhalation. In one embodiment, the glasses 104 and disposable face masks 106 could be available in numerous designs, constructions, styles, colors, and sizes. Exact size, measurement, construction, and design specifications may vary upon further development and manufacturing.

Referring to FIG. 4, an enlarged view of the disposable face mask 106 provided with the fastening members 110 in one embodiment is disclosed. In one embodiment, the fastening members 110 are placed on an outer surface at a top portion of the disposable face mask 100 as shown in FIG. 4. The fastening members 110 are configured to detachably secure the glasses 104 at the bottom to the disposable face mask 100. In one embodiment, the fastening members 110 could be, but not limited to, double-adhesive sticky tapes. In some embodiments, the fastening members 110 could be, but not limited to, a hook and loop fastener (Velcro®). In one embodiment, the disposable face masks 106 and the glasses 104 could be manufactured with different sizes that are suitable for males, females, and children. In some embodiments, the disposable face masks 106 and the glasses 104 could be manufactured in small, medium, and large sizes. In one embodiment, the glasses 104 could be designed in a stylish or a fashionable manner for public appeal, and color codes could be implemented for first responders, for example, red for firefighters, blue for medical personnel, black for law enforcement, green for armed forces, and clear for the general public.

It should be appreciated that the glasses can have other shapes and dimensions, and such variations are within the spirit and scope of the claimed invention. The glasses can also be configured to be used with corrective or prescriptive lenses, polarized lenses, or nonprescription lenses, such as with clear accessory lenses to alter the appearance or color, or to provide protection from ultraviolet (UV) rays (UV-A and UV-B type). The glasses can be comprised of materials such as standard plastic, high-index plastic, polycarbonate, trivex, hard resin (CR-39), and crown glass.

Preferred embodiments of this invention are described herein, including the best mode known to the inventors for carrying out the invention. It should be understood that the illustrated embodiments are exemplary only and should not be taken as limiting the scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings in the foregoing descriptions. Although specific terms may be employed herein, they are used only in generic and descriptive sense and not for purposes of limitation. Accordingly, the present invention is not limited to the specific embodiments illustrated herein.

1. A disposable semi-hardened face mask attached with glasses, comprising:
a disposable face mask formed of a material and shaped to be disposed across a portion of the face of a wearer in alignment with and surrounding the nose and mouth of the wearer;
a pair of glasses having a frame with bendable temple arms and lenses, wherein the frame is curved to ensure maximum fit and configured to be comfortably worn by the wearer for protecting eyes,
wherein the glasses further comprising an interior seal for preventing entry of airborne pathogens that may pass through the eyes from entering into the body;
one or more fastening members placed on an outer surface at a top portion of the disposable face mask,
wherein the fastening members are configured to detachably secure the glasses at the bottom to the disposable face mask, thereby forming and maintaining a sealed fit around the mouth, nose, and eyes to protect the wearer and preventing entry of airborne germs, bacteria, and viral and bacterial pathogens, which can be transmitted from human to human or animal to human or human to animal through saliva, nasal fluid or inhalation.

2. The disposable semi-hardened face mask attached with glasses of claim 1, wherein the disposable face mask having one or more vents for allowing exhaled air to easily escape from both sides of the disposable face mask, thereby preventing fogging of glasses due to moist and exhaled air that escapes from behind the mask.

3. The disposable semi-hardened face mask attached with glasses of claim 2, wherein the vents are at least any one of lightweight plastic or metal vents.

4. The disposable semi-hardened face mask attached with glasses of claim 1, wherein the glasses are trimmed around the lens region with at least any one of foam or a rubber material that will conform to the wearer's face.

5. The disposable semi-hardened face mask attached with glasses of claim 1, wherein the mask is made of a standard 3-ply cotton material.

6. The disposable semi-hardened face mask attached with glasses of claim 1, wherein the fastening members are at least any one of double-sided adhesive sticky tape and a hook and loop fastener. The disposable semi-hardened face mask attached with glasses of claim 1, wherein the glasses are secured at the bottom to the disposable face mask using double-adhesive sticky tapes.

8. The disposable semi-hardened face mask attached with glasses of claim 1, wherein the glasses are secured at the bottom to the disposable face mask using hook and loop fasteners.

9. A disposable semi-hardened face mask attached with glasses, comprising:
a disposable face mask formed of a material and shaped to be disposed across a portion of the face of a wearer in alignment with and surrounding the nose and mouth of the wearer, wherein the disposable face mask having one or more vents;
a pair of glasses having a frame with bendable temple arms and lenses, wherein the frame is curved to ensure maximum fit and configured to be comfortably worn by the wearer for protecting eyes,
wherein the glasses further comprising an interior seal for preventing entry of airborne pathogens that may pass through the eyes from entering into the body;
a double-sided adhesive sticky tape placed on an outer surface at a top portion of the disposable face mask,
wherein the double-sided adhesive sticky tape is configured to detachably secure the glasses at the bottom to the disposable face mask, thereby forming and maintaining a sealed fit around the mouth, nose, and eyes to prevent the entry of airborne germs, bacteria, and viral and bacterial pathogens, which can be transmitted from human to human or animal to human or human to animal through saliva, nasal fluid or inhalation and protect the wearer.

10. The disposable semi-hardened face mask attached with glasses of claim 9, wherein the vents are configured to allow exhaled air to easily escape from both sides of the disposable face mask, thereby preventing fogging of glasses due to moist and exhaled air that escapes from behind the mask.

11. The disposable semi-hardened face mask attached with glasses of claim 9, wherein the vents are at least any one of lightweight plastic or metal vents.

12. The disposable semi-hardened face mask attached with glasses of claim 9, wherein the glasses are trimmed around the lens region with at least any one of foam or a rubber material that will conform to the wearer's face.

13. The disposable semi-hardened face mask attached with glasses of claim 9, wherein the mask is made of standard 3-ply cotton material.

14. The disposable semi-hardened face mask attached with glasses of claim 9, wherein the glasses are secured at the bottom to the disposable face mask using hook and loop fasteners.

15. A disposable semi-hardened face mask attached with glasses, comprising:
a disposable face mask formed of a material and shaped to be disposed across a portion of the face of a wearer in alignment with and surrounding the nose and mouth of the wearer, wherein the disposable face mask having one or more vents for allowing exhaled air to easily escape from both sides of the disposable face mask;
a pair of glasses having a frame with bendable temple arms and lenses, wherein the frame is curved to ensure maximum fit and configured to be comfortably worn by the wearer for protecting eyes,
wherein the glasses are detachably secured at the bottom to the disposable face mask using fastening members, which are placed on an outer surface at a top portion of the disposable face mask, thereby forming and maintaining a sealed fit around the mouth, nose, and eyes to prevent the entry of airborne germs, bacteria, and viral and bacterial pathogens which can be transmitted from human to human or animal to human or human to animal through saliva, nasal fluid or inhalation and protecting the wearer.

16. The disposable semi-hardened face mask attached with glasses of claim 15, wherein the disposable face mask is made of a standard 3-ply cotton material.

17. The disposable semi-hardened face mask attached with glasses of claim 15, wherein the disposable face mask further comprises an interior seal for preventing entry of airborne pathogens that may pass through the eyes from entering into the body.

18. The disposable semi-hardened face mask attached with glasses of claim 15, wherein the vents prevent fogging of glasses due to moist and exhaled air that escapes from behind the mask, wherein the vents are at least any one of lightweight plastic or metal vents.

19. The disposable semi-hardened face mask attached with glasses of claim 15, wherein the glasses are trimmed around the lens region with at least any one of foam or a rubber material that will conform to the wearer's face.

20. The disposable semi-hardened face mask attached with glasses of claim 15, wherein the fastening members are at least any one of double-sided adhesive sticky tapes and hook and loop fasteners.
